(19) United States
(12) Patent Application Publication
(10) Pub. No.: US 2008/0230054 A1
(43) Pub. Date: Sep. 25, 2008

(54) Oropharyngeal Airway Device

(76) Inventor: Stephen Nicholas Prineas, New South Wales (AU)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 10/599,244

(22) PCT Filed: Mar. 23, 2005
(86) PCT No.: PCT/AU2005/000413

§ 371 Date: Apr. 1, 2008

(30) Foreign Application Priority Data
Mar. 23, 2004 (AU) 2004901578

(51) Int. Cl. A61M 16/00

(52) U.S. Cl. 128/200.26

(57) ABSTRACT

An oropharyngeal airway device (1) which includes a unitary tube (2) and a locating flange (6). The locating flange (6) forms, in combination with a first portion (4) of the tube (2), at least part of a mouthpiece (7) defining an inlet (8) to a passage (3). The flange (6) is adapted to locate adjacent an outer surface of the patient's mouth and the first portion (4) of the tube (2) extends into the mouth cavity. The tube (2) has a second portion (5), extending from said first portion (4), which has a distal end defining an outlet (12) to the tube (2) and which is adapted to extend to a location closely adjacent the base of the tongue. In use, the tube (2) is generally hook shaped with first portion (4) being substantially straight and the second portion (5) being of an arcuate form, extending obliquely from the first portion (4) and configured to follow the pharyngeal arc defined by the passage from the rear of the patient's mouth cavity through the oropharynx to a location adjacent the glottis. Alternatively or additionally to the tube shape described above, the outlet (12) at the distal end of the tube (2) can be defined by a first opening (13) that is configured to align with the opening to the larynx. Alternatively or additionally to the tube shape or the first opening described above, the device (1) can include internal markings (26) for guiding an endoscope therethrough.

### FIELD OF THE INVENTION

The present invention relates to improvements in oropharyngeal airway devices. The improvements relate to the ease of effective use of such devices and to assisting fibreoptic intubation with the airways in place.

A preferred form of the invention has been developed primarily for the purpose of facilitating nasal intubation and will be described in detail in relation to this particular use. However, it will be appreciated that the improved airway devices of the invention are also suited to use in basic airway management and for the more conventional procedure of intubation through the mouth.

### BACKGROUND OF THE INVENTION

The design of oropharyngeal airways in common use has remained largely unchanged for many years and appears not to have taken into account recent advances in airway management. In this regard the majority of devices commonly used for basic airway management, such as the “Guedel” airway, are not made long enough to reach beyond the base of the tongue, thereby often necessitating repeated manual intervention by the practioner in the form of manipulation of the patients head and chin to maintain a patient airway. It should also be noted that these devices are not in anyway designed to facilitate fibreoptic intubation.

Furthermore, other oropharyngeal devices that are specifically designed to assist fibreoptic intubation such as the “Berman” airway have a generally ‘J’ shaped profile which does not match well with the natural internal profile of the passages in a patient extending from the mouth opening through the oropharynx. This configuration makes it difficult to insert and accurately position these devices, as well as being unnecessarily uncomfortable for the patient. There is also a tendency with these ‘J’ shaped devices to depress the tongue, which may further contribute to creating an obstruction at the airway outlet. Furthermore, the inappropriate profile of these prior art devices means that the outlets of the devices may be directed at and into adjacent tissue rather than the internal body passages, which then makes it very difficult to accurately feed and guide an endoscope or the like through the device and beyond.

While there have been recent published developments relating to proposed adjustable telescopic oropharyngeal airway devices that have the capacity for extension of the outlet to the base of the tongue, these devices suffer from a number of inherent disadvantages. For example, the two part sliding construction is expensive, complicated to make and potentially difficult to operate. The telescopic arrangement also has the potential to create sealing problems around the mouthpiece. Furthermore, it is not easy to determine when these devices have been located at the optimal position and there is an increased risk the device could end up directed into the oesophagus under inexperienced hands. Also, the profile is again of a generally ‘J’ shaped form that is far from ideal. Furthermore, none of these prior art devices readily facilitate fibreoptic intubation via the nose as is often desirable when, for example, the patient is unconscious and/or has suffered severe facial trauma, or when surgery in or around the mouth is contemplated.

It is an object of the present invention to provide an oropharyngeal airway device that overcomes or ameliorates one or more of the disadvantages of the prior art or which at least provides a useful alternative.

### SUMMARY OF THE INVENTION

Accordingly, in a first aspect, the present invention provides an oropharyngeal airway device for location in a patient's mouth through the mouth cavity to maintain an unobstructed passageway extending from outside the patient's mouth to a position past a posterior aspect of the patient's tongue, said device including:
a unitary tube having a passage therethrough; and
a locating flange provided at a proximal end of a first portion of said tube to form, in combination with said first portion, at least part of a mouthpiece defining an inlet to said passage, the flange adapted for locating adjacent an outer surface of the patient's mouth when the first portion of the tube extends into the mouth cavity,
said tube having a second portion extending from said first portion, the second portion having a distal end which defines an outlet to the tube and which is adapted to extend to a location closely adjacent the base of the tongue,
wherein the tube is, in use, generally hook shaped with the first portion being substantially straight and the second portion being of an arcuate form, extending obliquely from the first portion and configured to follow the pharyngeal arc defined by the passage from the rear of the patient's mouth cavity through the oropharynx to a location adjacent the glottis.

The outlet at the distal end of the tube is preferably defined by a first opening that is configured to align with the opening to the larynx.

In a second aspect, the present invention provides an oropharyngeal airway device for location in a patient's mouth through the mouth cavity to maintain an unobstructed passageway extending from outside the patient's mouth to a position past a posterior aspect of the patient's tongue, said device including:
a unitary tube having a passage therethrough;
a locating flange provided at a proximal end of a first portion of said tube to form, in combination with said first portion, at least part of a mouthpiece defining an inlet to said passage, the flange adapted to locate adjacent an outer surface of the patient's mouth when the first portion of the tube extends into the mouth cavity,
said tube having a second portion extending from said first portion, the second portion having a distal end which defines an outlet to the tube and which is adapted to extend to a location closely adjacent the base of the tongue,
wherein the outlet at the distal end of the tube is defined by a first opening that is configured to align with the opening to the larynx.

Preferably, the tube is, in use, generally hook shaped with the first portion being substantially straight and the second portion being of an arcuate form, extending obliquely from the first portion and configured to follow the pharyngeal arc defined by the passage from the rear of the patient's mouth cavity through the oropharynx to a location adjacent the glottis.

The first opening is preferably defined by an end of the tube that is oblique to the axis of the tube so that the leading edge is adjacent the inside of the hook shaped formation.

In a first preferred form of the device specifically configured to facilitate nasal intubation with the airway in place, the second portion of the tube also includes a second opening in a posterior surface designed to align with the junction of the nasopharynx with the oropharynx which enables passage of an endoscope or the like into and through the device while it is in use. Preferably, this opening has a fusiform profile.

Desirably, this second posterior opening is fully surrounded by the tube material so as not to compromise the structural integrity of the tube at the outlet, but optionally also includes a resiliently openable split type formation that extends from this opening to the distal end of the tube ideally configured to enable easy removal of the airway device during intubation without again compromising the compression resistance of the tube adjacent the outlet. In a preferred form the edges of the split type formation are tapered obliquely to ease opening from the inside of the tube while resisting compression due to externally applied forces.

Preferably, the distal end of the tube also includes a protuberance configured to locate the device by engagement in the vallecula between the epiglottis and the back of the tongue. In a preferred form, the leading edge of the first opening forms the locating protuberance. More preferably this leading edge is rounded or swollen to exaggerate the locating formation and/or minimise trauma during insertion.

Ideally, the tube has a generally elliptical cross-section to optimise ease of insertion. Due to the arcuate shape of the device and in keeping with the common practice employed with more commonly used with more prevalent prior art devices, this device is designed to be passed upside down between the teeth initially. Once the distal opening has passed the teeth the device is rotated 180 degrees after which the device, guided by the curve of the tongue, slides with relative ease into its desired position. Occasionally unconscious patients resist mouth opening, thus desirably for a given cross-sectional area the tube should be wide and flat if the opening between the teeth is narrow. However this fusiform shape can apply torsional forces on the teeth when the device is rotated, especially when there is resistance to mouth opening. This could lead to damage to the teeth. A circular tube would minimise the forces on the teeth during rotation, but would require wider mouth opening. A tube which is elliptical in cross-section balances these two requirements.

When the outlet at the distal end of the tube aligned with the opening to the larynx, then in the majority of cases, this will result in the axis of the tube at the outlet being approximately perpendicular to the axis of the tube at the inlet or mouthpiece.

In a third aspect, the present invention provides an oropharyngeal airway device that is configured to include internal markings for the purpose of guiding an endoscope therethrough.

In a fourth aspect, the present invention provides an oropharyngeal airway device for location in a patient's mouth through the mouth cavity to maintain an unobstructed passageway extending from outside the patient's mouth to a position past a posterior aspect of the patient's tongue, said device including:
a unitary tube having a passage therethrough; and
a locating flange provided at a proximal end of a first portion of said tube to form, in combination with said first portion, at least part of a mouthpiece defining an inlet to said passage, the flange adapted to locate adjacent an outer surface of the patient's mouth when the first portion of the tube extends into the mouth cavity,
said tube having a second portion extending from said first portion, the second portion having a distal end which defines an outlet to the tube and which is adapted to extend to a location closely adjacent the base of the tongue,
wherein the device includes internal markings for the purpose of guiding an endoscope therethrough.

Preferably, the internal surface has a finish that is relatively non reflective and the markings formed to contrast with this finish. The markings can be, for example, by way of printing, embossing, or a combination of the same. The low reflective finish could be applied to the device as a coating or be inherent in the material from which the device is manufactured.

In the preferred form of the device that includes the second posterior opening, the tube includes markings that are positioned on the interior surface of the tube opposite the opening which then extend to the first opening and outlet of the passage at the distal end of the tube. The markings may be in the form of “runway” type markings configured to identify the central axis of that innermost surface of the device and may also be such as to indicate proximity from the outlet. In this manner an endoscope or bronchoscope, whether inserted through the mouth or via the nose and the second opening, can be guided so as to follow this surface which in turn will help direct the endoscope into the trachea as required.

Desirably, the periphery of all openings in at least the second portion of the tube will be similarly marked in contrast to the rest of the internal surface of the tube to make recognition of each location easier for the endoscopist.

The mouthpiece preferably include some means for providing rigidity that are adapted to prevent the patient biting down and blocking the passage. This means is suitable for all forms of the invention.

In those embodiments where the tube portion is produced from a relatively soft, flexible resilient material, the means for providing rigidity in the mouthpiece can include a reinforcing insert or attachment of another more rigid material.

The flange of the mouthpiece can be formed integral with the tube or the reinforcing insert as required and may also include hook formations or openings for the attachment of securing ties and the like. The mouthpiece preferably also includes a standard connector or connector mount to enable connection to an anaesthetic breathing circuit if required.

The device of the invention can be manufactured from any suitable biocompatible partially resilient material including the conventional polymeric materials currently used for these airways. However, the device may also be formed advantageously from a biocompatible shape memory alloy whereby the device is flexible at room temperature, but designed to conform to the preferred specially configured hook formation when heated to a predetermined temperature consistent with that expected in the patient's airway.

### BRIEF DESCRIPTION OF THE DRAWINGS

A preferred form of the invention will now be described, by way of an example only, with reference to the accompanying drawings in which:

FIG. 1 is a sectional side view illustrating an airway according to the present invention in the mouth of a patient;

FIG. 2 is a more detailed first sectional side view of the device shown in FIG. 1;

FIG. 3 is a front part sectional view of the device shown in the previous figures;

FIG. 4 is a top view of the previous illustrated device;

FIG. 5 is an underside part view of the device of the previous figures viewed from the posterior opening illustrating the internal markings;

FIG. 6 is a sectional side view illustrating the airway in use during nasal fibreoptic intubation; and

FIG. 7 is a sectional side view illustrating the airway in use during oral fibreoptic intubation.

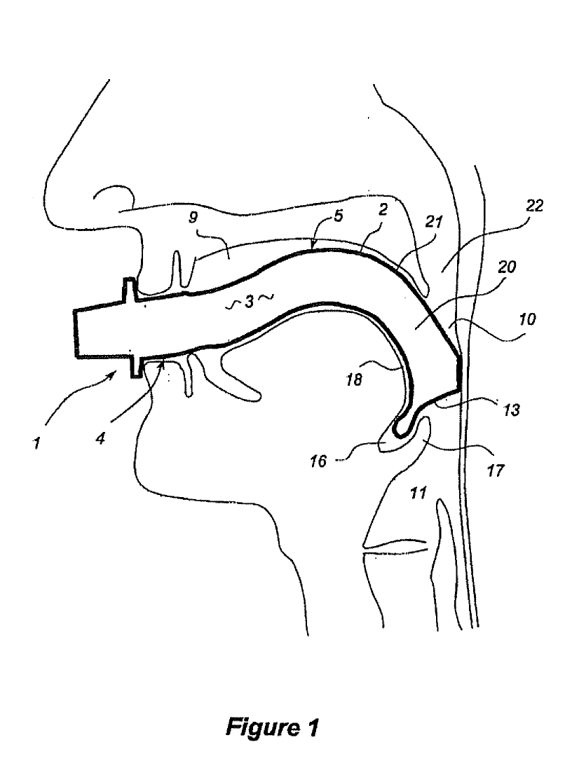

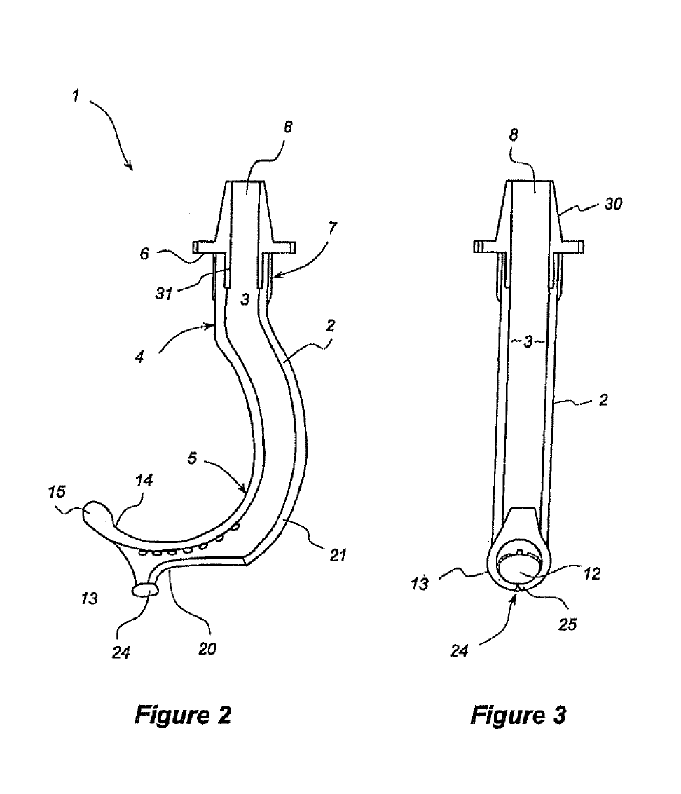

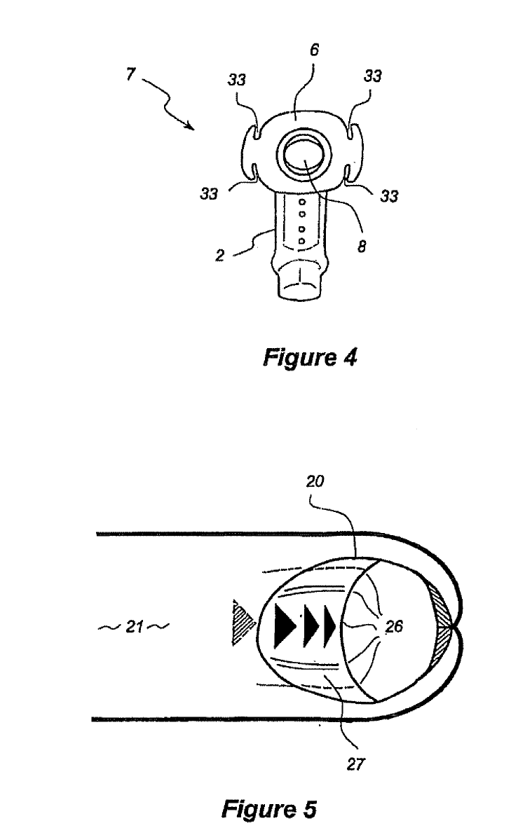

### DETAILED DESCRIPTION OF THE PREFERRED EMBODIMENTS

Referring to the drawings, there is shown an improved oropharyngeal airway device shown generally at 1. The device 1 includes a tube 2 having an open passage 3 extending therethrough.

The tube 2 comprises a first portion 4 and a second portion shown generally at 5. Connected to a proximal end of the first portion 4 is a flange 6 which in combination with the first portion 4 defines a mouthpiece shown generally at 7. The mouthpiece defines an inlet 8 to the passage 3.

The tube 2 has a generally elliptical section and is longitudinally configured such that, in situ within a patient's mouth, it has a generally hook shaped profile (and is thus distinct from some prior art devices that have a generally ‘J’ shaped profile). In this case the first portion 4 of the tube 2 is generally straight and the second portion 5 has an arcuate form that extends obliquely from the first portion. This second arcuate portion 5 is configured to closely follow the pharyngeal arc defined by the passage from the rear of the patient's mouth cavity 9 through the oropharynx 10 to a location adjacent the glottis 11.

The tube 2 has an outlet 12 at its distal end defined by a first opening referred to hereinafter as the laryngopharyngeal ring 13. This ring extends obliquely to the major axis of the tube and preferably has a rounded and raised ring lip 14.

The leading edge of the laryngopharyngeal ring 13 defines, by its oblique configuration, a locating protuberance 15 which may can be deliberately exaggerated or reconfigured to engage in the vallecula 16 between the epiglottis 17 and the back of the tongue 18. Ideally the ring lip 14 including the protuberance 15 is rounded or swollen to minimise trauma during insertion into the patient's mouth.

In the preferred illustrated form of the device that is specifically configured to facilitate nasal intubation with the airway in place, the device also includes a second, preferably fusiform, opening 20 in a posterior surface 21 of the second tube portion 5. This opening 20 is designed to align, in use, with the junction of the nasopharynx 22 with the oropharynx 10.

This second opening 20 is preferably fully surrounded by tube material so as not to compromise the structural integrity of the first opening 13 defined by the ring lip 14. However, the device also includes a resiliently openable split type of formation 24 which extends from the second opening through to the first opening. The edges 25 of the split 24 are tapered obliquely to ease opening from the inside of the tube while resisting compression due to externally applied forces.

Preferably, the internal surface of at least the second portion of the tube is formed from or coated with a relatively non-reflective material and markings are provided thereon for guiding a fibreoptic device through the airway. In the preferred illustrated form, the markings 26 are provided at a location generally opposite the second opening 20 on the innermost interior surface 27 and extend right through to the first opening 13.

Preferably, the markings are configured to guide an endoscopist or the like to the interior mid-line of the device, like the markings on an airport runway.

Finally, the device 1 also includes a combined connector/mouthpiece reinforcing insert shown generally at 30. This insert has a mouthpiece reinforcing spigot 31 and a standard male connecting spigot 32 preferably sized for connection to a standard 15 mm female connector for attachment of an anaesthetic breathing circuit as required. Although the illustrated form has the flanges 6 formed as part of the connector 30, an alternative would be to form the flanges integrally with the tube 2. The illustrated flange 6 can also include hook formations 33 for attaching ties or the like for securing the mouthpiece to the patient.

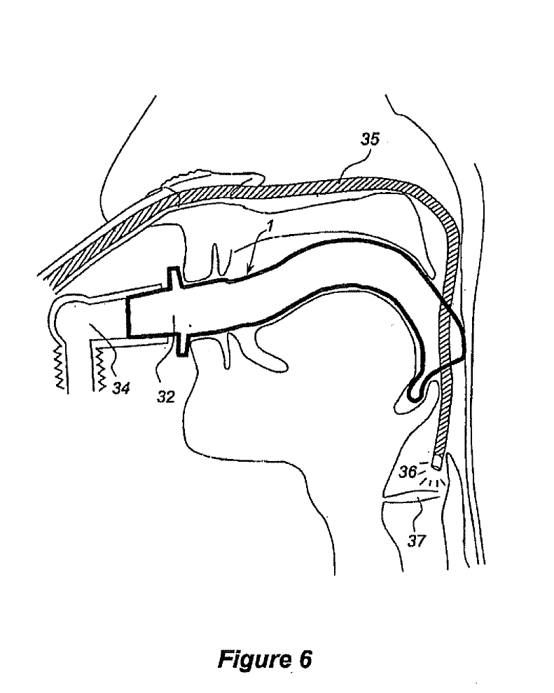

The use of the device 1 will now be described. The device is designed to be inserted in a manner familiar to most doctors, nurses and paramedics already trained in resuscitation and the use of “Guedel” type airways. Dentures if present should be removed. The device 1 should be well lubricated with a water-based gel. With the mouth open, the device 1 is passed between the teeth and gums in an upside down orientation, i.e. with the protuberance 15 of the ring lip 14 being disposed closer to the upper teeth/gums and the second opening 20 closer to the lower teeth/gums. Once the ring lip 14 is passed the teeth, the device 1 is then rotated 180° and should slide along the tongue into the pharynx with minimal resistance. The mouthpiece 7 should come to rest comfortably between the teeth. Oxygen and other gases can then be delivered to the patient either by a standard face mask or by attaching a standard 15 mm female anaesthetic breathing circuit connector 34 to the connector mount 32 as shown in FIG. 6.

The generally hook shaped profile of the device 1 advantageously conforms to the anatomy of the patient, which minimises trauma to the patient. Also, the laryngopharyngeal ring 13 locates adjacent the glottis and forms a ring of support at the back of the larynx and supports the tongue. This advantageously maintains an open air passage by supporting soft tissue which could otherwise sag or droop into occluding the patient's airway. This type of airway occlusion can be particularly problematic in devices which are non-anatomical in shape.

Asleep fibreoptic intubation in spontaneously breathing patients is a technique that may be indicated in patients with a predicted high likelihood of difficult intubation and in whom other options, such as an awake fibreoptic intubation or awake tracheostomy, are not possible or desirable.

After pre-oxygenation and, if required, installation of the upper airway with local anaesthetic, the patient is sent to sleep by gradual increments of an inhalational or intravenous anaesthetic agent, so that spontaneous ventilation is maintained. Once a light plane of anaesthesia is achieved it should be possible to insert the device 1 in the manner described above. The anaesthetic circuit is connected and oxygen given while the anaesthesia is deepened. The airway can be maintained by basic manoeuvres such as jaw thrust or head tilt and if needed adhesive transparent dressings can be used to form a seal around the device at the mouth.

As shown in FIG. 6, once sufficient depth of anaesthesia is achieved, an intubating fibreoptic bronchoscope 35 with a lubricated endotracheal tube already ‘railroaded’ over it can be passed through the nose to the back of the pharynx. If the bronchoscope is kept in the midline the endoscopist should encounter the ‘runway’ markings 26 immediately in front. The endoscopist can then follow the runway markings to the entrance of the larynx 36 and the vocal cords 37.

Once past the cords, the endotracheal tube can be passed over the bronchoscope 35, through the nose to the back of the pharynx. At this point the device 1 is advantageously able to be pulled easily from around the bronchoscope 35 and out the mouth, whilst leaving the bronchoscope 35 in place to best allow passage of the endotracheal tube into the larynx. The bronchoscope 35 passes through the split 24 during removal of the device 1.

It is also possible to pass the endotracheal tube through the ring of the device and into the trachea, but this is not recommended because of the risk of tearing the cuff of the endotracheal tube at the ring.

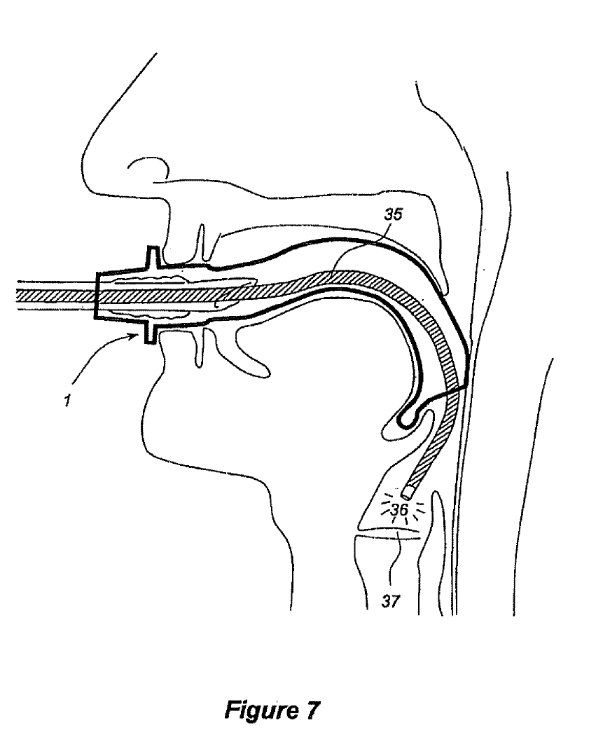

Use of the device 1 to facilitate oral intubation is very straightforward as shown in FIG. 7.

It will be clear from the above that the preferred form of the invention provides a simple easy to use device which unlike the prior art is ideal for both basic airway management as well as readily facilitating both nasal and oral intubation if required.

It will also be appreciated from the above, that regardless of the specific configuration of the preferred form of the device as previously described, there are huge operational advantages in simply having an oropharyngeal airway device that is visually configured to facilitate fibreoptic intubation by providing an internal surface of a distinct but low reflective finish with prominent markings thereon as required. At present, the majority of these devices are manufactured from a semitranslucent white polymeric material which is highly reflective and no contrasting markings are provided.

Furthermore, while preferred forms of the invention will most likely be manufactured from the commonly presently used bio-compatible polymeric materials that are resilient but of a pre-formed shape at room temperature and elevated temperatures, the use of bio-compatible shape memory alloys is also contemplated. The alloys would be selected to have reasonable flexibility at room temperature to aid in assertion with a view to reforming into the preferred preformed hook shape configuration once at body temperature after insertion.

In another variation (not shown), the airway device can include a removable covering over the second opening 20 to assist with intubation through the mouth if required.

Alternatively, if the device is intended to be used solely for intubation through the mouth, it can be formed without the second opening 20 or the split 24.

Accordingly, while the invention has been described in reference to specific embodiments and variations, it will be appreciated that the invention can be embodied in many other forms.

1. An oropharyngeal airway device for location in a patients's mouth through the mouth cavity to maintain an unobstructed passageway extending from outside the patient's mouth to a position past a posterior aspect of the patient's tongue, said device including:
a unitary tube having a passage therethrough;
a locating flange provided at a proximal end of a first portion of said tube to form, in combination with said first portion, at least part of a mouthpiece defining an inlet to said passage, the flange adapted to locate adjacent an outer surface of the patient's mouth when the first portion of the tube extends into the mouth cavity,
said tube having a second portion extending from said first portion, the second portion having a distal end which defines an outlet to the tube and which is adapted to extend to a location closely adjacent the base of the tongue,
wherein the outlet at the distal end of the tube is defined by a first opening that is configured to align with the opening to the larynx; and
wherein the second portion of the tube also includes a second opening in a posterior surface, the second opening adapted to align with the junction of the nasopharynx with the oropharynx.

2. The device as claimed in claim 1, wherein the tube is in use, generally hook shaped with the first portion being substantially straight and the second portion being of an arcuate form, extending obliquely from the first portion and configured to follow the pharyngeal arc defined by the passage from the rear of the patient's mouth cavity through the oropharynx ta a location adjacent the glottis.

3. The device as claimed in claim 2, wherein the first opening is defined by an end of the tube that is oblique to the axis of the tube so that the leading edge is adjacent the inside of the hook shaped formation.

4. The device as claimed in claim 1, wherein the second opening has a fusiform profile.

5. The device as claimed in claim 1, wherein the second posterior opening is fully surrounded by the tube material.

6. The device as claimed in claim 1, wherein the second posterior opening includes a resiliently openable split type formation that extends from the second posterior opening to the distal end of the tube.

7. The device as claimed in claim 6, wherein the edges of the split type formation are tapered obliquely.

8. The device as claimed in claim 1, wherein the distal end of the tube also includes a protuberance configured to locate the device by engagement in the vallecula between the epiglottis and the back of the tongue.

9. The device as claimed in claim 1, wherein the distal end of the tube also includes a protuberance configured to locate the device by engagement in the vallecula between the epiglottis and the back of the tongue and the leading edge of the first opening forms the locating protuberance.

10. The device as claimed in claim 9, wherein the leading edge is rounded or swollen.

11. The device as claimed in claim 1, wherein the axis of the tube at the outlet is approximately perpendicular to the axis of the tube at the inlet or mouthpiece.

12. The device as claimed in claim 1, wherein the external wall of the tube is generally elliptical when viewed in transverse cross-section.

13. An oropharyngeal airway device that is configured to include internal markings for the purpose of guiding an endoscope therethrough.

14. An oropharyngeal airway device for location in a patient's mouth through the mouth cavity to maintain an unobstructed passageway extending from outside the patient's mouth to a position past a posterior aspect of the patient's tongue, said device including:
a unitary tube having a passage therethrough; and
a locating flange provided at a proximal end of a first portion of said tube to form, in combination with said first portion, at least part of a mouthpiece defining an inlet to said passage, the flange adapted to locate adjacent an outer surface of the patient's mouth when the first portion of the tube extends into the mouth cavity,
said tube having a second portion extending from said first portion, the second portion having a distal end which defines an outlet to the tube and which is adapted to extend to a location closely adjacent the base of the tongue,
wherein the device includes internal markings for the purpose of guiding an endoscope therethrough.

15. The device as claimed in claim 14, wherein the internal surface has a finish that is relatively non reflective and the markings formed to contrast with this finish.

16. The device as claimed in claim 14 wherein the markings are made by way of printing, embossing or a combination of the same.

17. The device as claimed in claim 15, wherein the low reflective finish is applied to the device as a coating.

18. The device as claimed in claim 15, wherein the low reflective finish is inherent in the material from which the device is manufactured.

19. The device as claimed in claim 1, wherein the tube includes markings, for the purpose of guiding an endoscope therethrough, that are positioned on the interior surface of the tube opposite the second opening which then extend to the first opening and outlet of the passage at the distal end of the tube.

20. The device as claimed in claim 19, wherein the markings are in the form of “runway” type markings configured to identify the central axis of that innermost surface of the device.

21. The device as claimed in claim 20, wherein the markings are adapted to indicate proximity from the outlet.

22. The device as claimed in claim 19, wherein the periphery of all openings in at least the second portion of the tube will be similarly marked in contrast to the rest of the internal surface of the tube.

23. The device as claimed in claim 1, wherein the mouthpiece includes means for providing rigidity that are adapted to prevent the patient biting down and blocking the passage.

24. The device as claimed in claim 23, wherein the tube portion is produced from a relatively soft, flexible resilient material and the means for providing rigidity in the mouthpiece include a reinforcing insert or attachment of another more rigid material.

25. The device as claimed in claim 1 wherein the flange of the mouthpiece is formed integral with the tube or the reinforcing insert.

26. The device as claimed in claim 25, wherein the flange includes hook formations or openings for the attachment of securing ties.

27. The device as claimed in claim 25, wherein the mouthpiece includes a standard connector or connector mount to enable connection to an anaesthetic breathing circuit if required.

28. The device as claimed in claim 1, wherein the device is manufactured from a biocompatible partially resilient material.

29. The device as claimed in claim 1, wherein the device is formed from a biocompatible shape memory alloy whereby the device is flexible at room temperature, but conforms to a hook formation when heated to a predetermined temperature consistent with that expected in the patient's airway.
